(19) United States
(12) Patent Application Publication
(10) Pub. No.: US 2008/0193793 A1
(43) Pub. Date: Aug. 14, 2008

(54) Material For Doped And Undoped Hole And Electron Transport Layer

(75) Inventors: Hans-Hermann Johannes, Braunschweig (DE); Wolfgang Kowalsky, Braunshweig (DE); Guergana Dobreva, Braunschweig (DE); Martin Duemeland, Halle (DE); Ulf Lawrentz, Furth (DE)

Correspondence Address: OBLON, SPIVAK, MCCLELLAND MAIER & NEUSTADT, P.C., 1940 DUKE STREET, ALEXANDRIA, VA, 22314, US

(73) Assignees: Technische Universitaet Braunschweig, Braunschweig (DE); BASF Aktiengesellschaft, Ludwigshafen (DE)

(21) Appl. No.: 11/722,599

(22) PCT Filed: Dec. 23, 2004
(86) PCT No.: PCT/EP04/53704

§ 371 Date: Nov. 19, 2007

(51) Int. Cl. H01L 51/30; C07C 255/42; C07C 211/54

(52) U.S. Cl. 428/690; 564/434; 558/395; 438/99; 257/E51.024

(57) ABSTRACT

The present invention relates to materials useful as an electron or hole transport or injection layer in organic electrooptic devices. The material can form or be part of an electrically conductive organic layer, suitable for the transport of so-called positive charges or holes. The inventive HTL compounds are intrinsically doped HTLs which allows their deposition with greater homogeneity and reproducibility than matrix compositions consisting of a matrix material and an admixed p-dopant.

The present invention relates to materials useful as a hole or electron transport layer or as a dopant for hole or electron transport layers as well as for hole or electron injection layers in organic electric, preferably electrooptic devices. The material usable as a hole or electron transport layer (HTL and ETL, respectively) or a hole or electron injection layer can form or be part of an electrically conductive organic layer, suitable for the transport of so-called positive charges or holes. Electrooptic devices for the purposes of this disclosure comprise organic light emitting diodes (OLEDs), organic field effect transistors (OFETs), lasers and photovoltaic devices suitable for photovoltaic solar energy conversion.

### STATE OF THE ART

In general, electrooptic devices comprise a plurality of electrically conductive organic compounds which are stacked in layers which are arranged between electrodes.

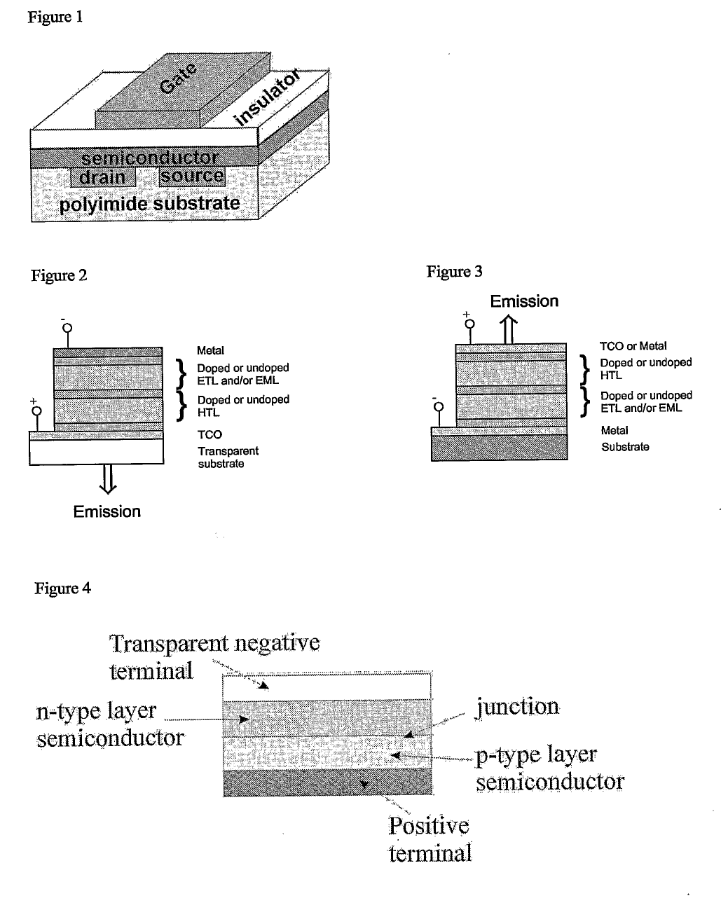

The general structure of EL devices, which the inventive material for a hole transport layer can be utilized is depicted schematically in FIGS. 1 to 4.

In the case of OLEDs, adjacent the anode, consisting for example of ITO (indium tin oxide), there is a hole transport layer (HTL), optionally with an intermediate hole injection layer (HIL). Next to the HTL, an emissive layer is arranged, the compounds of which generally emit visible light, with the energy stemming from an exciton generated by the simultaneous localization of an electron and a hole on the same molecule within the emissive layer, transport layer and adjacent the emissive layer, there is arranged an electron transfer layer and, subsequently, a cathode (for example Mg, LiF/Al, Ca, Ba).

Optionally, electron transport beyond the emissive layer towards the anode may be prevented by an electron blocking layer arranged between the hole transport layer and the emissive layer. As a further option, the migration of holes beyond the emissive layer towards the cathode may be prevented by a hole blocking layer arranged between the emissive layer and the electron transfer layer.

There may be arranged an electron injection layer between the electron transfer layer and the cathode. Further, the electron transfer layer or electron injection layer may be separated from the cathode by an electron conductive protective layer in order to allow the processing steps necessary for applying the cathode onto the electron injection layer.

Materials for hole transport layers (HTL) are known from WO 2004/016711 A1, for example α-NPD as an intrinsic HTL or m-MTDATA, p-doped with F₄-TCNQ.

Zhou et al. (Applied Physics Letters, Vol. 78, No. 4, pages 410 to 412) disclose OLEDs using a p-doped amorphous hole injection layer. A hole transport layer of both polycrystalline phthalocyanines and amorphous 4, 4′, 4′ tris-(N,N-diphenylamine)triphenylamine (TDATA) p-doped by co-evaporation with F₄-TCNQ (tetrafluoro-tetracyano-quinodimethane) was shown to yield a conductivity orders of magnitude above that of undoped matrix materials. Zhou et al. show that p-doping of the matrix material leads to a larger current density at lower voltages applied as well as to maximum electroluminescence (EL) efficiencies at lower driving voltages.

As a result, OLEDs containing p-doped HTLs exhibit a very low operating voltage and an improved EL efficiency as a result of controlled doping.

### OBJECTS OF THE INVENTION

It is an object of the present invention to provide an alternative to p-doped HTL materials for use in organic electrooptic devices.

In its preferred embodiment, the present invention seeks to provide HTL materials suitable for organic electrooptic devices that have improved characteristics, like an increased stability at elevated temperatures.

It is a further object of the invention to provide a method for synthesis of novel HTL materials.

### GENERAL DESCRIPTION OF THE INVENTION

The present invention achieves the above-mentioned objects by providing a material suitable for a hole and electron transport and/or injection layer in organic electrooptic devices according to the following general formula I:

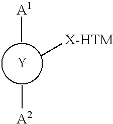

In general formula I, moiety Y represents a central carbon based structure, optionally comprising hetero atoms, for example having 5 to 14 atoms, conjugatedly linking accessory residues A¹ and A². Residues A¹ and A² are electron accepting residues, having at least one π-bond, and are conjugated through moiety Y to form a conjugated or aromatic system. In addition to accessory residues A¹ and A², further electron donating groups may be conjugatedly linked to moiety Y. In addition to residues A¹ and A², a hole transport moiety (HTM) or more HTMs are covalently linked to moiety Y by intermediate moiety X. The at least one HTM is capable of hole transporting electric charges by the mechanism known as hole transport. However, the at least one HTM is not conjugated linked to moiety Y.

Intermediate moiety X can be a chemical bond or any carbon atom and/or heteroatom comprising moiety suitable to non-conjugatedly link the at least one HTM to Y.

The number of HTMs ranges from at least one to a maximum that moiety Y is capable of non-conjugatedly linking. As an example for moiety Y being a five- or six-membered carbon ring, optionally substituted with heteroatoms, the number of HTMs can be 1, 2, 3 or 4. The number of residues X and of HTM can increase when moiety Y consists of one, two or more condensed rings, e.g. comprising a total of 5 to 14 carbon atoms or heteroatoms.

Examples for moiety Y are shown in the following embodiments of general formula I, wherein the designations refer to moiety Y derivatized with Xs, A¹ and A²:

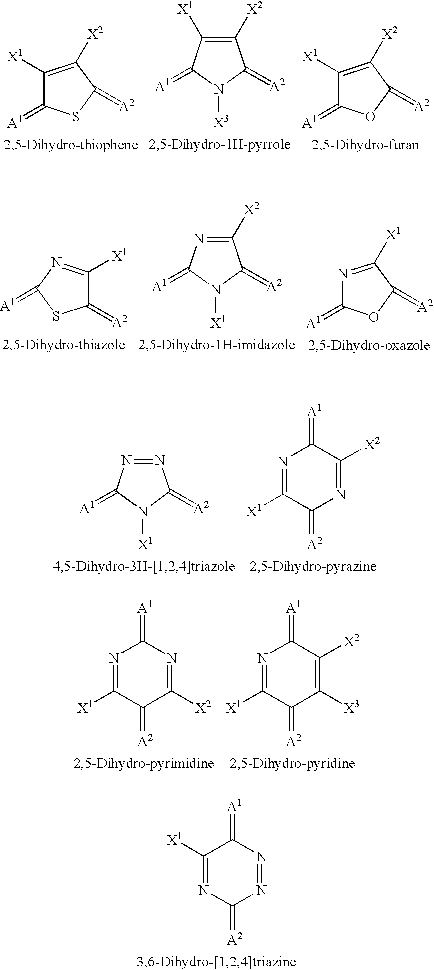

Further embodiments of general formula I are represented by compounds of formulae II to VI:

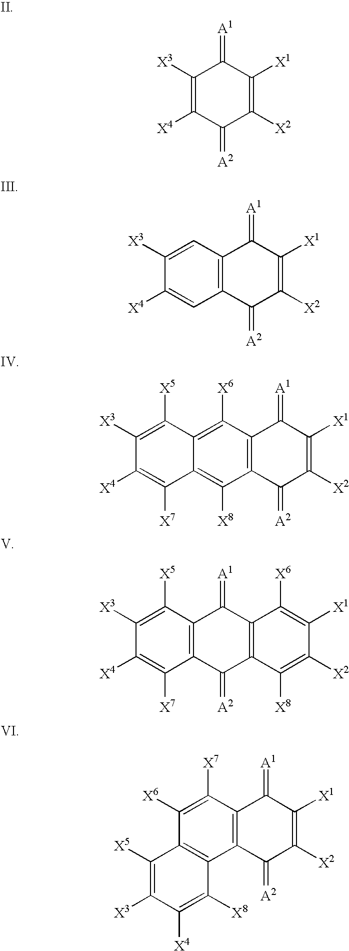

In the above formulae embodying general formula I, X is exemplified as X¹, X², X³, X⁴ to X⁸, respectively, which are each independently intermediate groups or atoms or a chemical bond, at least one of which forms the intermediate moiety that is substituted with an HTM.

In formulae I to VI, the central carbon based conjugated or aromatic moiety Y may comprise heteroatoms, e.g. N, O, S, Si, Ge, replacing one or more carbon atoms.

Residues A¹ and A² are electron acceptor residues, having at least one π-electron rich bond, capable of enhancing the density of π-electrons within moiety Y. In addition to accessory residues A¹ and A², further electron donating groups may be conjugatedly linked to moiety Y. In addition to residues A¹ and A², a hole transport moiety (HTM) or more HTMs are covalently linked to moiety Y by intermediate moiety X. The at least one HTM is capable of hole or electron transport. However, the at least one HTM is not conjugatedly linked to moiety Y.

Embodiments of general formulae II to VI are preferred compounds, wherein HTM is selected from the group comprising moieties capable of hole transport, for example tris-[(N,N-diaryl)amino]-triphenylamines like 4,4′,4″-tris[(N-(1-naphthyl)-N-phenyl-amino-triphenylamine] (1-TNATA) and its derivatives, 4,4′,4″-tris[(N-(2-naphthyl)-N-phenyl amino)-triphenylamine] (2-TNATA) or 4,4′,4″-tris[(N-(3-methylphenyl)-N-phenyl-amino)-triphenylamine] (m-TDATA) and its derivatives, 4,4′,4″-tris(carbazole-9-yl)triphenylamines; N,N,N′,N′-tetra arylbenzidines as N,N,N′,N′-tetra phenyl benzidine and its derivatives, N,N′-bis(1-naphthyl)-N,N′-diphenyl-benzidine (α-NPD), N,N′-di(naphthalene-2-yl)-N,N′-diphenyl-benzidine (β-NPD), 4,4′-bis(carbazole-9-yl)biphenyl (CBP) and its derivatives, and their heteroatom substituted analogs (e.g. thienyl-, selenyl-, furanyl-derivatives); 4,4′-bis(2,2′-diphenylvinyl)-1,1′-biphenyl (DPVBI); triarylamines and their derivatives, 4,4′-bis(N,N-diarylamino)-terphenyls, 4,4′-bis(N,N-diarylamino)-quarterphenyls and their homologs and derivatives;

wherein A¹ and A² are independently electron donator moieties, for example selected among cyano groups, —C(CN)₂, —NCN;

wherein at least one of X¹ to X⁸ is chemically bonded to a hole transport moiety (HTM). The at least one of X¹ to X⁸ can be selected from the group comprising —O—, —S—, R—, —SiR¹R², —CR¹R², —CR¹═CR², —NR¹, —N═CR¹, —N═N—, and a chemical bond;

wherein X¹ to X⁸, which are not bonded to an HTM, can be selected independently from the group comprising —H, —F, —CN, R—, —OR¹, —SR¹, —NR¹R², —SiR¹R²R³, —CR¹R²R³, —CR¹═CR²R³, —N═NR¹, and HTM;

wherein R, R¹, R² and R³ can be selected independently from substituted or unsubstituted alkyl, vinyl, allyl and/or (hetero-)aryl and/or (hetero-) cyclic moieties, hydrogen or HTM as defined above.

When the X binding the at least one HTM to Y is a chemical bond, the compound according to the invention is a dye.

It is a specific advantage of the HTL or ETL compounds according to the present invention that they are intrinsically p-doped and, accordingly, a co-evaporation for building an HTL of a matrix material in combination with its dopant is no longer necessary. Accordingly, the production process for organic EL devices is facilitated using the HTL compounds according to the present invention. As a further effect of the intrinsically doped HTL compounds according to the present invention, HTLs are more homogenous and can be deposited with greater reproducibility in comparison to matrix compositions consisting of a matrix material and an admixed p-dopant.

A further advantage of the HTL and ETL compounds according to the invention are their higher glass transition temperatures in comparison to the system of p-doped matrix materials. The higher glass transition temperatures are assumed to result from steric effects within the HTL compounds.

A further characteristic of the HTL and ETL compounds according to the invention is their generally reduced mobility within an electric field, which is a desired property for constructing stable organic electroluminescent (EL) devices, preferably resulting in an increased long-term stability.

The inventive compounds are intrinsically doped HTLs and ETLs, respectively, which allows their deposition with greater homogeneity and reproducibility than matrix compositions consisting of a matrix material and an admixed dopant.

In a further aspect, the present invention provides a method for production of the HTL compounds, comprising the following central synthetic steps:

A) A di-substituted 1,4-cyclohexanedione moiety is reacted to a stable diketal, for example a cyclic ketal on both ketone moieties. The two substituting groups of the 1,4-cyclohexanedione moiety are chemically reactive to allow the formation of a chemical bond to an HTM. Preferred substituting groups are π-electron rich compounds, for example the substituent groups can suitably be halogenated aromatic moieties that are reactive with HTM comprising an aromatic residue.

The linkage with at least one HTM is obtained by reaction of the chemically reactive substituent group with a residue of the HTM, for example by reaction of a halogenated phenyl group with an aromatic residue of the HTM. The linkage can be direct between the 1,4-cyclohexanedione moiety and the HTM or via intermediate linker moieties. As a result, the cyclohexyl moiety, substituted with two diketal groups, is derivatized on its reactive substituent groups with HTMs. In a subsequent oxidation reaction, the two diketal groups are reoxidized to ketone groups.

It is essential, that the at least one HTM is linked in a non-conjugated manner with the 1,4-cyclohexanedione moiety.

B) Following the linkage of at least one HTM to the cyclohexyl moiety substituted with two opposing ketone groups, the ketones are reacted for replacement of the ketones by electron acceptor moieties, for example cyano imine groups or a dicyano methylene group. A subsequent oxidation generates a 1,4-cyclohexanedione group, conjugated in positions 2 and 5 to two electron donating moieties. The 1,4-cyclohexanedione group is additionally substituted non-conjugatedly with at least one HTM.

In general, HTL compounds according to the present invention can be coated according to known techniques, including vapour deposition (including PVD, CVD, OVPD) and coating (spray, spin or knife coating) from a solution or sputtering, depending on the molecular weight and solubility of the compounds. In general, HTL compounds having a very high molecular weight are difficult to evaporate and, accordingly, in such cases coating from a solution is preferred. A person skilled in the art can easily determine an appropriate method for coating HTL compounds. Methods for the determination of an appropriate solvent are also commonly known. Preferred solvents are chlorobenzene, toluene and xyloles.

The present invention will now be described by way of examples, which are not intended to limit the scope of the invention. Reference is made to the figures, wherein

FIG. 1 schematically depicts an organic field electric transistor (OFET) in cross-section. Therein, the layer designated as semiconductor illustrates the position of an HTL according to the invention,

FIG. 2 schematically depicts an OLED in cross-section with the HTL being formed of the compounds according to the present invention,

FIG. 3 schematically depicts an inverted OLED in cross-section with the HTL in form of the compounds according to the invention,

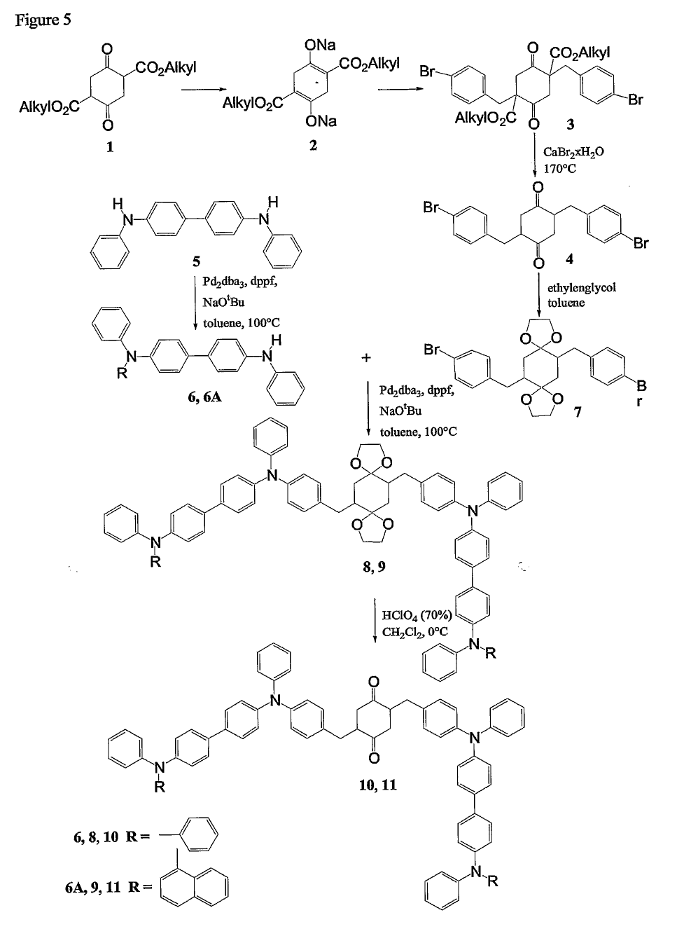

FIG. 4 schematically depicts a solar cell in cross-section with the p-type layer semiconductor being formed of a compound according to the present invention,

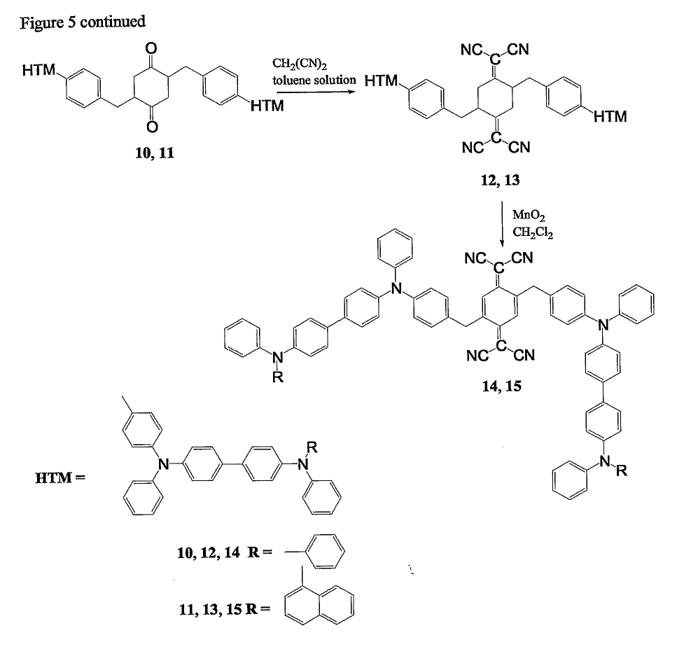

FIG. 5 schematically shows the steps for synthesis of exemplary compounds according to the invention,

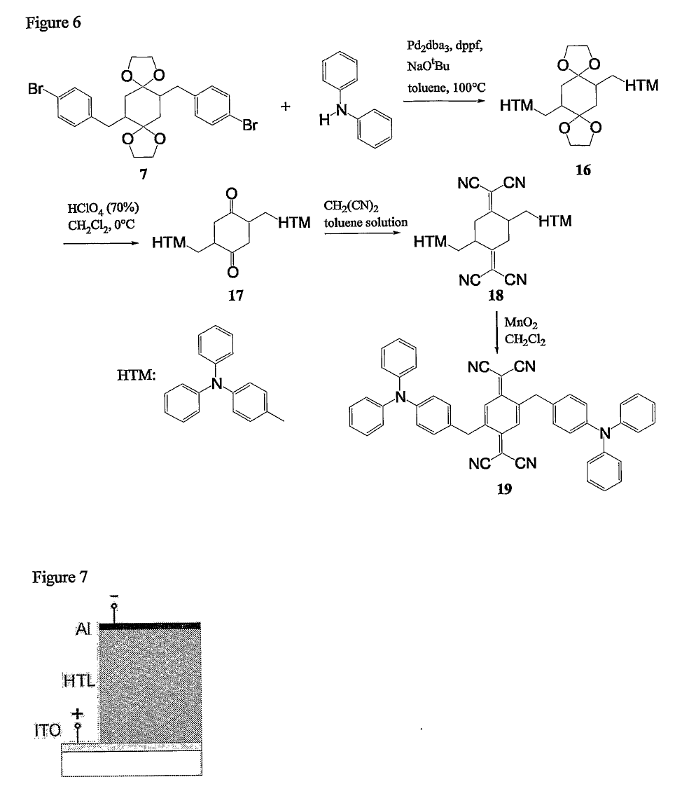

FIG. 6 schematically shows the steps for synthesis of inventive compound 19,

FIG. 7 schematically shows the structure comprising an inventive compound as a charge transport layer,

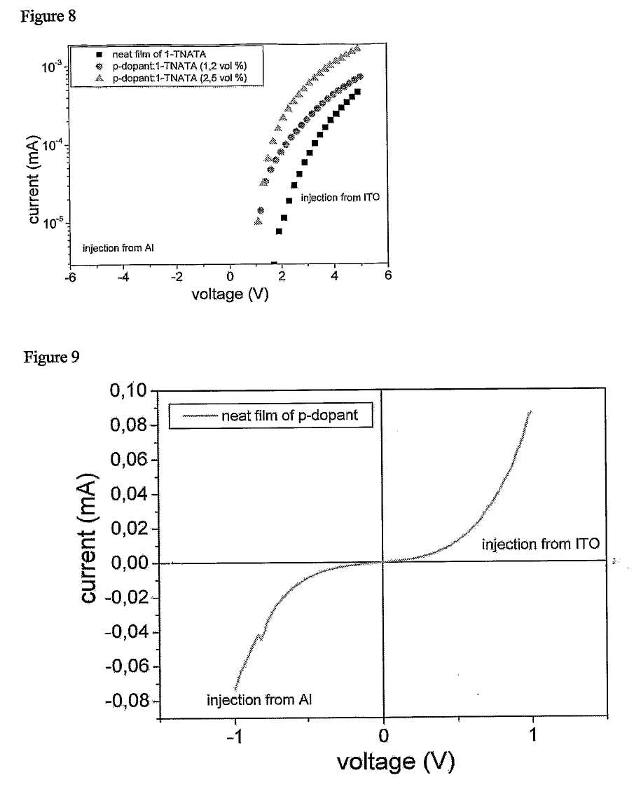

FIG. 8 shows the electrical behaviour of 1-TNATA in comparison to 1-TNATA doped with inventive compound 19, and

FIG. 9 shows the electrical behaviour of inventive compound 19.

### EXAMPLE 1

### OLED Comprising Inventive HTL, Vacuum Deposited

This example describes the structure of an inverted OLED, schematically depicted in FIG. 3, however, a non-inverted structure, e.g. schematically depicted in FIG. 2, can be realized using the compounds according to the invention as well. For a non-inverted OLED structure, compound 14 and/or compound 19, obtainable according to Example 3, was vacuum deposited onto an ITO covered glass substrate up to a layer thickness of 10-500 nm. Subsequently, an electron blocking layer was vacuum deposited, followed by vacuum deposition of an emissive layer (Alq₃), a hole blocking layer (BCP) and an electron transport layer (TAZ). The cathode (LiF/Al) was deposited as the final layer.

For an inverted OLED structure, after deposition of a cathode on a substrate, an electron transport layer, followed by an optional hole blocking layer, an electroluminescent layer and an optional electron blocking layer, the inventive compound was deposited to form the hole transport layer as a dopant in 1-TNATA or as a pure substance. Deposition of the hole transport layer was followed by vacuum deposition of a protective layer (pentacene) before deposition of poly(3,4-[ethylenedioxy]-thiophene) (PEDT) with poly(styrene sulfonic acid) (PSS), also known as PEDT:PSS (e.g. Baytron P®), before applying ITO as the cathode.

In the alternative to a hole transport layer comprising the compound according to the invention as a dopant, the inventive compound can form the hole transport layer as the only component of this layer.

Accordingly, in a further embodiment, compound 14 and/or 19 can form an injection layer, superseding the need for PEDT:PSS as an injection layer. As a consequence, the pentacene protective layer can be omitted. This represents a specific advantage of the compounds according to the invention, especially because the injection layer can be applied in a vacuum process, e.g. without interrupting the vacuum processing to coat the final electrode layer. Further, the specific advantage of the compounds according to the invention to obviate the need for an injection layer (e.g. PEDT:PSS) and a protective layer (e.g. pentacene) allows for a more simple structure of the structure of the organoelectric device. Prior to the invention, the protective layer was required to allow the coating of the stacked sensitive organoelectric compounds using wet-chemical processing to allow the coating of highly conductive compounds like PEDT:PSS.

### EXAMPLE 2

### OLED Comprising Inventive HTL, Deposited by Spin Coating

Example 1 was repeated except that compound 14, or alternatively compound 19, obtained according to example 3, was deposited by spin coating from a solution in (solvent) to a final layer thickness of 10-500 nm.

The electric and EL properties essentially corresponded to those found in Example 1.

### EXAMPLE 3

### Electric Properties of 2,5-bis-(4-diphenylaminobenzyl)-1,4-bis-(dicyanomethylidene)-cyclohexa-2,5-diene (19)

The synthesis of compound 19 is schematically shown in FIG. 6. Synthesis of compound 19 was essentially analogous to synthesis of 14, described in example 4. The melting point of compound 19 is 252° C.

As an example for the compounds according to the invention, compound 19, shown in FIG. 6, was used as a p-dopant within a 100 nm thickness layer of 1-TNATA at concentrations of 1.2 vol-% and 2.5 vol-%, respectively in comparison to undoped 1-TNATA. 1-TNATA doped with compound 19 was coated onto ITO-covered glass substrate and covered by an electrically conductive aluminum layer.

The structure of the measuring set-up, and of a simple electroorganic device, respectively, are depicted in FIG. 7. As can be taken from FIG. 7, the inventive compounds can form a layer in direct contact with both electrode surfaces without any need for an additional injection layer when used as a dopant in admixture with 1-TNATA or as a pure substance, i.e. without additional matrix material. This structure is sufficient for transporting charge from one electrode to the other.

The electric properties of a charge transport layer comprising an inventive substance as a dopant of matrix material (1-TNATA) are shown in FIG. 8, demonstrating that increasing concentrations of compound 19 result in a dramatically increased positive charge transport (I[A]) in response to increasing voltage (U[V]), i.e. when charge was injected from the ITO layer. When voltage was reversed to inject charge from the A1 layer, no conductivity was measured.

As an alternative to using the inventive compound as a p-dopant, the electrically conductive layer was formed of compound 19 (neat film of p-dopant) by itself within a structure according to FIG. 7, i.e. without further matrix compounds in admixture. For measurements, the polarity of electrodes was reversed if necessary. Compound 19 was layered onto ITO glass substrate and covered with aluminum as above. When applying voltage to inject charge from the ITO layer, the response is a rapid increase in positive charge transport, whereas reversing the voltage to inject charge from the Al layer results in a rapid increase of negative charge transport. This behaviour, shown in FIG. 9, is proof for the suitability of the inventive compounds for forming electrically conductive layers in FETs.

This example demonstrates that compounds according to the invention can form a p-dopant in hole transport layers and, alternatively, that they can form hole transport layers and electron transport layers without further matrix materials added. Further, this is proof for the applicability of the inventive substances as hole and/or electron injection layers. As a specific advantage, a conductive layer formed of the compounds according to the invention yields a very homogenous and evenly distributed phase.

### EXAMPLE 4

### Synthesis of 2,5-bis{4-[(4′-diphenylamino-biphenyl-4-yl)-phenylamino]-benzyl}-1,4-bis(dicyanomethyliden)-cyclohexa-2,5-diene (14)

The synthesis of 2,5-bis{4-[(4′-diphenylamino-biphenyl-4-yl)-phenylamino]-benzyl}-1,4-bis(dicyanomethyliden)-cyclohexa-2,5-diene (14) is schematically depicted in FIG. 5. In this example, the 4′-bromobenzyl substituents represent the two substituting groups of the 1,4-cyclohexanedione moiety that are chemically reactive to allow the later formation of a chemical bond to an HTM.

Starting from 2,5-bis(methoxycarbonyl)-cyclohexa-1,4-dione (1), 2,5-bis(methoxycarbonyl)-2,5-bis(4-bromobenzyl)-cyclohexa-1,4-dione (3) is accessible via intermediate compound 2, by reacting it with 4-bromobenzylbromide. Analytical results for compound 3 are MS (EI, 70 eV): m/z (%)=566 (10) [M⁺], EA: calc. C=50.91, H=3.91, Br=28.22; measured C=51.25, H=3.95, Br28.07.

Removal of the two ester groups in 2- and 5- positions is obtained by heating compound 3 in the presence of calcium bromide. In detail, 2,5-bis(4-bromobenzyl)cyclohexa-1,4-dione (4) is obtained by stirring 1.13 g (2 mmol) 2,5-bis(methoxycarbonyl)-2,5-bis(4-bromobenzyl)-cyclohexa-1,4-dione (3) in mixture with 2.80 g calcium bromide at 180° C. under an inert gas atmosphere in a 250 mL three-necked flask equipped with a reflux condenser for 3 hours. Then, 100 mL of 1 N HCl were added. Phases were separated and the aqueous phase was extracted four times with 15 mL methylene chloride. The organic phase is dried over sodium sulfate and the solvent is removed in a rotary evaporator. The solid product is purified by Soxhlet extraction using diethyl ether. 0.72 g (1.6 mmol) of a white solid are obtained as the isomeric mixture. Analytical results for 4 are MS (EI, 70 eV): m/z (%)=450 (40) [M⁺]

EA: calc. C=53.36, H=4.03, Br=35.50; measured C=53.74, H=4.07, Br=35.45, melting point m.p.=158-160° C.

The conversion of 4 to 2,5-bis(4-bromobenzyl)-1,4,9,12-tetraoxa-dispiro[4.2.4.2]tetradecane (7) was performed according to Liebigs Ann. Chem. 186-190 (1982). MS (EI, 70 eV): m/z (%)=538 (16) [M⁺]. EA: calc. C=53.55, H=4.87, Br=29.69; measured C=55.33, H=4.94, Br=29.10, melting point m.p.=200-202° C.

As a representative HTM, N,N′,N′-triphenyl-biphenyl-4,4′-diamine (6) is obtainable from compound 5 via the Buchwald coupling, using a reaction in toluene at 100° C. according to J. Am. Chem. Soc. 118, 7215-7216 (1996) and J. Am. Chem. Soc. 62, 1568-1569 (1997). Compound 6 is purified by flash column chromatography (n-hexane:ethyl acetate (10:1), Rf=0.3).

2,5-bis{4-[4′-diphenylamino-biphenyl-4-yl)-phenyl-amino]-benzyl}-1,4,9,12-tetraoxa-dispiro[4.2.4.2]tetradecane (8) is isolated as a white solid from the reaction of 6 with 7. The melting point of 8 was determined to 142-143° C. ESI-MS (CH₃CN/toluene): 1201 [M⁺]. Elementary analysis (EA): calc. C=83.97, H+6.04, N=4.66; measured C=83.87, H=6.12, N=4.42.

In the alternative, 2,5-bis(4-{[4′-(naphthalene-1-yl-phenyl-amino)-biphenyl-4-yl]-amino}-benzyl)-1,4,9,12-tetraoxa-dispiro[4.2.4.2]tetradecane (9) is obtained as a white solid from the reaction of 6A with 7. The melting point was determined to 130-132° C. ESI-MS (CH₃CN/toluene): m/z=1301 [M⁺]

EA: calc. C=84.88, H=5.90, N=4.30; measured C=84.72, H=6.24, N=3.78.

2,5-bis(4-{[4′-diphenylamino-biphenyl-4-yl]-phenylamino]-benzyl}-cyclohexane-1,4-dione (10) was obtained by cooling 0.6 g (0.5 mmol) of compound 8, dissolved in 70 mL dichloromethane in a 250 mL two-necked flask under inert gas atmosphere to 0° C. Slowly, 0.5 mL 70% HClO₄ are added dropwise and stirring continued at 0° C. for one hour. After 3 hours, the reaction solution is neutralised with 100 mL saturated NaHCO₃ and stirred for one hour at room temperature. The organic phase is dried over magnesium sulfate and the solvent is removed on a rotary evaporator. The residue is purified by flash column chromatography in methylene chloride:n-hexane (5:1). 0.185 mg (1.6 mmol) of a white solid are obtained as an isomeric mixture having a melting point of 142° C. and 136° C., respectively.

Isomer 2: ¹H NMR (200 MHz, CDCl₃): δ=7.36-6.87 (m, 54H, H_{ar.}), 3.18 (dd, J=13.9 and 4.2 Hz, 2H, H_{cyc.}), 3.09-2.71 (m, 2H, H_{cyc.}), 2.62 (dd, J=17.7 and 5.9 Hz, 2H, H_{cyc.}), 2.49-2.21 (m, 4H, H_{methylene}).

¹³C NMR (50 MHz, CDCl₃): δ=209.3 (C_{C═O}), 147.7-146.4 (Cₐᵣ.), 134.8-122.8 (C_{ar.}), 48.3 (C_{cyc., CH}), 41.4 (C_{cyc.,CH2}), 34.5 (C_{methylene}).

ESI-MS (CH₃CN/toluene): m/z=1112 [M⁺]

In the alternative to compound 10, 2,5-bis(4-{[4′-(naphtalene-1-yl-phenylamino)-biphenyl-4-yl]-phenylamino}-benzyl)-cyclohexa-1,4-dione (11) is obtained by the same synthetic steps as compound 10 above when starting from compound 9 instead of compound 8. Compound 11 is obtained as a white solid (isomeric mixture).

Compounds 10 and 11, respectively, are reacted to 2,5-bis-{4-[(4′-diphenylamino-biphenyl-4-yl)-phenylamino]-benzyl}-1,4-bis-(dicyanomethylidene)-cyclohexane (12) and 2,5-bis-(4-{[4′-(naphthalene-1-yl-phenylamino)-biphenyl-4-yl)-phenylamino}-benzyl)-1,4-bis-(dicyanomethylidene)-cyclohexane (13) by stirring 0.9 mmol of compound 10 and 11, respectively, (0.99 g compound 10) in mixture with 0.178 g (2.7 mmol) CH₂(CN)₂ and a catalytic amount of beta-alanine 10 mL alcoholic toluene solution, with the alcohol preferably being methanol, ethanol or propanol, in a 50 mL one-necked flask, equipped with a reflux condenser. After 72 hours of stirring at 80° C., compounds 12 and 13, respectively, are removed by filtration and washed with ethanol. There are obtained 0.85 g (0.7 mmol) of compound 12 as a pale yellow solid having a melting point of 278-280° C.

Compound 12: ¹H NMR (200 MHz, CDCl₃): δ=7.48-7.01 (m, 54H, Hₐᵣ.), 3.71 (dd, 2H, H_{cyc.}), 3.22 (d, 2H, H_{cyc.}), 2.79-2.61 (m, 6H, H_{methylene and cyc.}).

¹³C NMR (50 MHz, CDCl₃): δ=176.9 (C_{C═C(CN)}), 147.9-146.5 (Cₐᵣ.), 135.5-123.0 (C_{ar.}), 110.9 (C_{CN}), 110.7 (C_{CN}), 88.5 (C_{C═C(CN)}), 45.6 (C_{cyc., CH}), 39.9 (C_{cyc., CH2}), 35.4 (C_{methylene}). ESI-MS (CH₃CN/toluene): m/z=1208 [M⁺]

EA: calc. C=85.40, H=5.33, N=9.26; measured C=84.92, H=5.32, N=8.90

Compound 13 is isolated as a yellow solid having a melting point of 156-158° C.

Compound 13: ¹H NMR (200 MHz, CDCl₃): δ=7.97-7.76 (m, 7H, H_{methylene}),7.52-6.90 (m, 53H, Hₐᵣ.), 3.68 (dd, 2H, H_{cyc.}), 3.21 (d, 2H, H_{cyc.}), 2.84-2.70 (m, 6H, H_{methylene and cyc.}). ¹³C NMR (50 M, CDCl₃): δ=176.9 (C_{C═c(CN)}), 148.5-1143.6 (Cₐᵣ.), 135.6-122.0 (C_{ar.}), 111.0 (C_{cn}), 110.7 (C_{CN}), 88.4 (C_{C═C(CN)}), 45.6 (C_{cyc., CH),} 40.0 (C_{cyc., CH2}), 34.1 (C_{methylene}). ESI-MS (CH₃CN/toluene): m/z=1309 [M⁺].

EA: calc. C=86.21, H=5.23, N=8.56; measured C=86.10, H=5.45, N=7.80

### 2,5-bis{4-[(4′-diphenylamino-biphenyl-4-yl)-phenylamino]-benzyl)-1,4-bis-(dicyanomethylidene)-cyclohexa-2,5-diene (14)

In a 250 mL two-necked flask there are dissolved 0.2 g (0.2 mmol) of compound 12 in 50 mL of dichloromethane. To the resultant solution, 0.55 g activated manganese dioxide is added. The mixture is stirred at room temperature under inert gas atmosphere for 3 h and is then filtered over silica gel. The solvent is removed and the residue is recrystallized from a mixture of toluene and acetonitrile at −10° C. There are obtained 0.12 g (0.1 mmol) of compound 14 as a green solid with a melting point of 177-179° C.

¹H NMR (400 MHz, CDCl₃): δ=6.99-7.46 (m, 56H, H_{cyc and ar.}), 4.27 (s, br., 4H, H_{methylene}), ¹³C NMR (100 MHz, CDCl₃): δ=150.3 (C_{C═C(CN)}), 147.7-122.8 (Cₐᵣ), 143.6 (C_{cyc., C═CH}), 124 (C_{cyc., c═CH}), 112.7 (C_{CN}) 113.9 (C_{CN}), 87.1 (C_{C═C(CN)}), 38.4 (C_{methylene}). ESI-MS (CH₃CN/toluene): m/z=1204 [M⁺]. Elementary analysis: calc.: C=85.69, H=5.02, N=9.30; measured: C=85.54, H=5.56, N=8.54.

1. A compound according to formula I 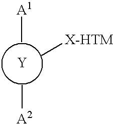
wherein moiety Y represents a central carbon based structure with accessory residues A¹ and A² each conjugatedly linked to moiety Y, accessory residues A¹ and A² are electron accepting residues, HTM is a hole transport moiety which is non-conjugatedly linked to moiety Y, and X is one or more intermediate groups, or a chemical bond.

2. The compound according to claim 1, wherein X is a chemical bond.

3. The compound according to claim 1, wherein moiety Y comprises a five- or six-membered ring which conjugatedly links accessory residues A¹ and A².

4. The compound according to claim 3, wherein the five- or six-membered ring comprises at least heteroatom.

5. The compound according to claim 1, wherein formula I is selected from the following formulae:

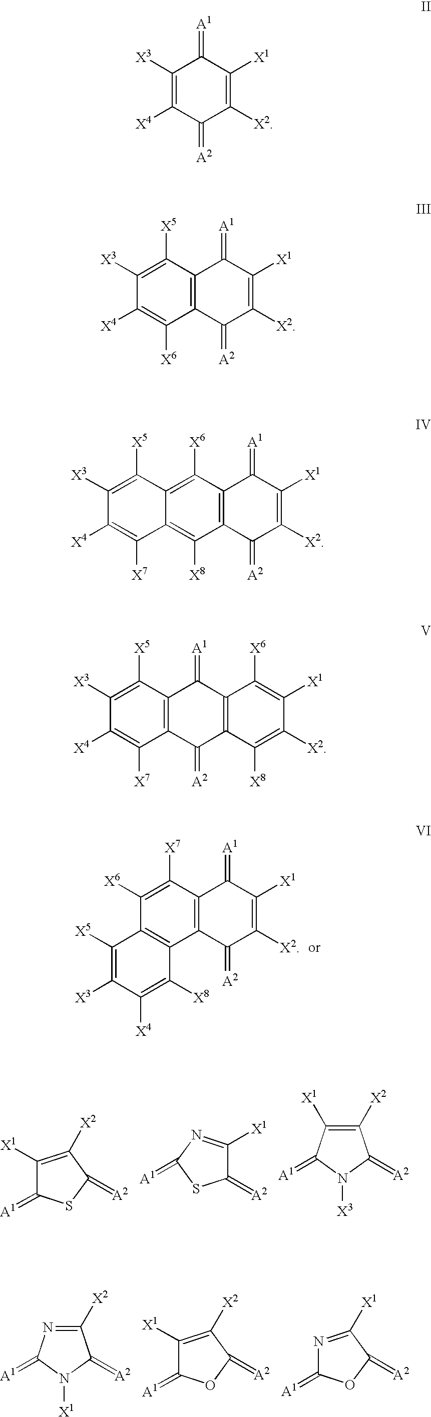

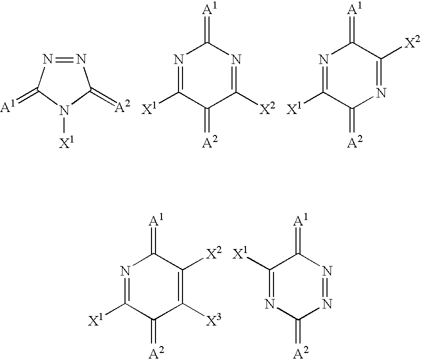

6. The compound according to claim 1, wherein from 1 to 8 HTM are present and each HTM is linked to one of X¹, X², X³, X⁴ to X⁸.

7. The compound according to claim 6, wherein X¹, X², X³, X⁴ to X⁸ which are substituted with HTM are each independently selected from the group comprising —O—, —S—, —SiR²R², —CR¹R², —CR¹═CR², —NR¹, —N═CR¹, —N═N—, and a chemical bond.

8. The compound according to claim 6, wherein X¹, X², X³, X⁴ to X⁸ which are not substituted with HTM are each independently selected from the group comprising —H, —F, —CN, —OR¹, —SR¹, —NR¹R², —SiR¹R²R³, —CR¹R²R³, —CR¹═CR²R³, —N═NR¹, and HTM.

9. The compound according to claim 1, wherein HTM is selected from the group comprising tris-[(N,N-diaryl)amino]-triphenylamines, 4,4′,4″-tris[(N-(1-naphthyl)-N-phenyl-amino-triphenylamine] (1-TNATA) and its derivatives, 4,4′,4″-tris[(N-(2-naphthyl)-N-phenyl amino)-triphenylamine] (2-TNATA); 4,4′,4″-tris[(N-(3-methylphenyl)-N-phenyl-amino)-triphenylamine] (m-TDATA) and its derivatives, 4,4′,4″-tris(carbazole-9-yl)triphenylamines; N,N,N′,N′-tetra arylbenzidines as N,N,N′,N′-tetra phenyl benzidine and its derivatives, N,N′-bis(1-naphthyl)-N,N′-diphenyl-benzidine (α-NPD), N,N′-di(naphthalene-2-yl)-N,N′-diphenyl-benzidine (β-NPD), 4,4′-bis(carbazole-9-yl)biphenyl (CBP) and its derivatives, 4,4′-bis(2,2′-diphenylvinyl)-1,1′-biphenyl (DPVBI); triarylamines and their derivatives, 4,4′-bis(N,N-diarylamino)-terphenyls, 4,4′-bis(N,N-diarylamino)-quarterphenyls and their homologs and derivatives.

10. The compound according to claim 6, wherein the HTM is a heteroatom substituted analog thereof.

11. The compound according to claim 10, wherein the heteroatom substituted analog is a thienyl-, selenyl- or furanyl-derivative.

12. The compound according to claim 1, wherein accessory resides A¹ and A² are each independently selected from the group comprising cyano groups, —C(CN)₂, and —NCN.

13. The compound according to claim 1, wherein the compound is 2,5-bis {4-[(4′-diphenylamino-biphenyl-4-yl)-phenylamino]-benzyl)-1,4-bis-(dicyanomethylidene)-cyclohexa-2,5-diene.

14. The compound according to claim 1, wherein the compound is 2,5-bis-(4-diphenylaminobenzyl)-1,4-bis-(dicyanomethylidene)-cyclohexa-2,5-diene.

15. A dopant in a hole or electron transport layer and/or in a hole or electron injection layer comprising a compound according to claim 1.

16. A hole or electron transport layer and/or a hole or electron injection layer consisting essentially of a compound according to claim 1.

17. A process for synthesizing a compound according to claim 1, wherein said process comprises
a) reacting a di-substituted 1,4-cyclohexanedione moiety to a stable diketal,
b) forming a non-conjugated chemical bond between the 1,4-cyclohexanedione moiety to at least one HTM,
c) chemically replacing the ketone groups for electron donating moieties to form a conjugated bond between the electron donating moieties and the cyclohexyl moiety, and
d) oxidizing 1,4-bis(dicyanomethylidene)-cyclohexane to 1,4-bis(dicyano-methylidene)-1,4-cyclohexa-2,5-diene.

18. An electric device comprising a compound according to claim 1.

19. The electric device according to claim 18, wherein the electric device is an organic light emitting diode (OLED), an organic field electric transistor (OFET), a laser or a photovoltaic device.

20. A process for producing an electric or electrooptic device, wherein said process comprises coating the electric or electrooptic device with the compound according to claim 1.

21. A process for producing an electric or electrooptic device, wherein said process comprises vacuum depositing the compound according to claim 1 on the electric or electrooptic device.

22. The process according to claim 21, wherein said vacuum depositing is a PVD (physical vapour deposition), CVD (chemical vapour deposition) or an OVPD (organic vapour physical deposition) process.

23. The process according to claim 20, wherein said coating is from solution or sputtering.

24. The process according to claim 23, wherein said coating is spray, spin, dip or knife coating.
